(19) United States
(12) Patent Application Publication
(10) Pub. No.: US 2019/0374508 A1
(43) Pub. Date: Dec. 12, 2019

(54) THERAPEUTIC TOPICAL COMPOSITIONS OF APREMILAST

(71) Applicant: SARUDBHAVA FORMULATIONS PRIVATE LIMITED, Hyderabad, Telangana (IN)

(72) Inventor: Venkata Nookaraju Sreedharala, Hyderabad, Telangana (IN)

(21) Appl. No.: 16/481,257

(22) PCT Filed: Jan. 24, 2018
(86) PCT No.: PCT/IN2018/050038

§ 371 Date: Jul. 26, 2019

(30) Foreign Application Priority Data
Jan. 27, 2017 (IN) 201741003041

(51) Int. Cl. A61K 31/4035; A61K 9/00; A61P 17/06

(52) U.S. Cl. CPC A61K 31/4035; A61K 45/06; A61P 17/06; A61K 9/0014

(57) ABSTRACT

The present invention relates to topical compositions comprising apremilast. It also relates to processes for preparing such compositions and methods of using them in management of skin diseases or disorders such as psoriasis, dermatosis, eczema, rosacea, acne vulgaris, allergies, contact and atopic dermatitis, pruritus, seborrhea, skin cancers, inflammation and other associated skin conditions.

### CROSS-REFERENCE TO RELATED APPLICATIONS

This application is a National Stage application of International Patent Application No. PCT/IN2018/050038, filed on Jan. 24, 2018, which claims priority to Indian Patent Application No. 201741003041, filed on Jan. 27, 2017, each of which is hereby incorporated by reference in its entirety.

### TECHNICAL FIELD

The application provides formulations for the topical administration of apremilast. The present application also provides processes for preparing such compositions and methods of using them in management of skin diseases or disorders such as psoriasis, dermatosis, eczema, rosacea, acne vulgaris, allergies, contact and atopic dermatitis, pruritus, seborrhea, skin cancers, inflammation and other associated skin conditions.

### BACKGROUND

Apremilast is a phosphodiesterase4 (PDE4) inhibitor. Apremilast is known chemically as N-[2-[(1S)-1-(3-ethoxy-4-methoxyphenyl)-2-(methylsulfonyl)ethyl]-2,3-dihydro-1,3-dioxo-1H-isoin-dol-4-yl]acetamide, having chemical structure as given below:

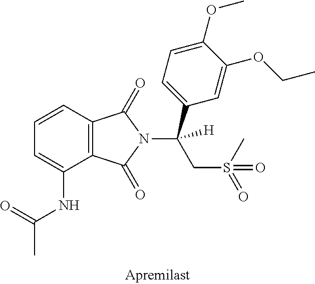 [SMILES: CCOc1cc([C@@H](CS(C)(=O)=O)N2C(=O)c3cccc(NC(C)=O)c3C2=O)ccc1OC]

Apremilast is at present available only as an oral formulation, marketed by Celgene Corp, under the trade name of OTEZLA®. OTEZLA® tablets are supplied in 10, 20, and 30 mg strengths for oral administration. OTEZLA® tablets are indicated (1) for the treatment of patients with active psoriatic arthritis and (2) for the treatment of patients with moderate to severe psoriasis who are candidates for phototherapy or systemic therapy.

Psoriasis, a chronic, idiopathic, genetically determined, and environmentally influenced disorder has been treated empirically for centuries with varied success. Its association with arthropathy and its significant psychological consequences have necessitated a comprehensive team approach for optimal care. Psoriasis is a non-contagious autoimmune disease which affects the skin and joints. It commonly causes red scaly patches to appear on the skin. The scaly patches caused by psoriasis, called psoriatic plaques, are areas of inflammation and excessive skin production. Skin rapidly accumulates at these sites and takes on a silvery-white appearance. Plaques frequently occur on the skin of the elbows and knees, but can affect any area including the scalp and genitals. In contrast to eczema, psoriasis is more likely to be found on the extensor aspect of the joint. The disorder is a chronic recurring condition which varies in severity from minor localized patches to complete body coverage. Fingernails and toenails are frequently affected (psoriatic nail dystrophy) and can be seen as an isolated finding. Psoriasis can also cause inflammation of the joints, which is known as psoriatic arthritis. Ten to fifteen percent of people with psoriasis have psoriatic arthritis. The cause of psoriasis is not known, but it is believed to have a genetic component. There are many treatments available, but because of its chronic recurrent nature psoriasis is a challenge to treat.

According to the National Institutes of Health, psoriasis is one of the most common human skin disorders, affecting greater than 3% of the United States population, or more than 5 million adults, of which greater than 1.5 million are considered to have a moderate to severe form of the disease. Although psoriasis is not fatal, it negatively impacts quality of life to a degree similar to heart disease and arthritis (Rapp et al. 1999). In addition, 10-30% of patients with psoriasis also develop a form of arthritis-psoriatic arthritis, which damages the bone and connective tissue around the joints. Furthermore, inflammatory mediators associated with psoriasis may increase the risk of obesity, diabetes, thrombosis, and atherosclerosis (Davidovici et al. 2010).

Rosacea is a chronic condition which is characterized by facial erythema and occasionally red domed papules and pustules. Four subtypes (Phymatous, Erythematotelangiectatic, Papulopustular, and Ocular) and three variants (conglobate, fulminans, and phymatous) of rosacea have been identified. Since there is no specific test for rosacea, it is generally diagnosed by visual inspection and it is currently treated with oral and topical antibiotics, alpha-hydroxy acid peels and dermatological laser treatment (N. Scheinfeld, T. Berk, “A Review of the Diagnosis and Treatment of Rosacea” Postgraduate Medicine 2010, 122(1): 139-143.) Rosacea has no cure, and lifelong treatment for relief of symptoms is often necessary (B. Culp, N. Scheinfeld, “Rosacea: A Review” Pharmacy & Therapeutics 2009, 34(1): 38-45.)

The term “eczema” refers to a set of clinical observations; it is commonly used as a generic term for various forms of dermatitis, including atopic and contact dermatitis. Recent studies have shown that a member of the cathelicidin family of host defense peptides, LL-37, is expressed in small amounts in humans but accumulates in skin affected by diseases such as atopic dermatitis (P. Y. Ong et al., “Endogenous Antimicrobial Peptides and Skin Infections in Atopic Dermatitis” N Engl. J. Med. 2002, 347(15): 1151-1160) rosacea (K. Yamasaki et al., “Increased serine protease activity and cathelicidin promotes skin inflammation in rosacea” Nature Med. 2007, 13:975-980) and psoriasis. In addition, the IL-19 cytokine is reportedly over-produced in psoriasis patients (E. Witte et al., “IL-19 Is a Component of the Pathogenetic IL-23/IL-17 Cascade in Psoriasis” J. Invest. Dermatol. 2014, 134:2757-2767.) Evidence is also mounting for the involvement of mast cells in rosacea (Y. Muto et al., “Mast Cells Are Key Mediators of Cathelicidin-Initiated Skin Inflammation in Rosacea” J. Invest. Dermatol. 2014, 134:2728-2736.)

Though, apremilast is available as an effective oral therapy for psoriasis; an oral tablet composition is less suitable for patients who have difficulty in swallowing or whose gastro intestinal side effects are not reduced even after suggested oral dose titration. Further, evidence from clinical trials and postmarketing experience suggestsan association between suicidal ideation and behavior with the use of apremilast. In clinical trials, an imbalance of cases of depression in patients treated with apremilast was also identified.

Thus, there is need for an effective pharmaceutical composition of apremilast suitable for topical administration that overcomes the problems associated with oral compositions. Use of a topical formulation is advantageous in that it avoids first-pass metabolism, circumvents gastrointestinal (“GI”) absorption, can allow delivery of an active ingredient with a relatively short biological half-life, and/or a narrow therapeutic window and facilitates uniform plasma dosing of the active ingredient

Further, there is an unmet need for improved patient compliant topical formulations that are effective in the treatment of skin disorders, and which provide improved delivery of the active agent, apremilast, at the desired site of action, with decreased inconvenience and irritation, increased ease of use for the patient, and longer duration of action.

### SUMMARY

The present application provides pharmaceutical compositions comprising apremilast as the active agent, suitable for topical administration.

Thus in one embodiment, the present application provides stable topical compositions comprising a therapeutically effective amount of apremilast, a pharmaceutically acceptable carrier and one or more pharmaceutically acceptable excipients.

In another embodiment, the present application provides pharmaceutical compositions comprising apremilast as the active agent, in any dosage form suitable for topical administration. Preferably, the compositions are in a form such as a solution, suspension, dispersion, emulsion, cream, ointment, gel, lotion, foam, paste or spray.

In yet another embodiment, the present application provides uses of topical compositions comprising apremilast as the active agent, for prophylaxis, amelioration, or treatment of skin diseases or disorders such as psoriasis, dermatosis, eczema, rosacea, acne vulgaris, allergies, contact and atopic dermatitis, pruritus, seborrhea, skin cancers, inflammation and other associated skin conditions.

In another embodiment, the application provides processes for preparing pharmaceutically stable topical compositions comprising apremilast.

### BRIEF DESCRIPTION OF THE DRAWINGS

FIG. 1: Absolute Ear Thickness measurements (in mm)

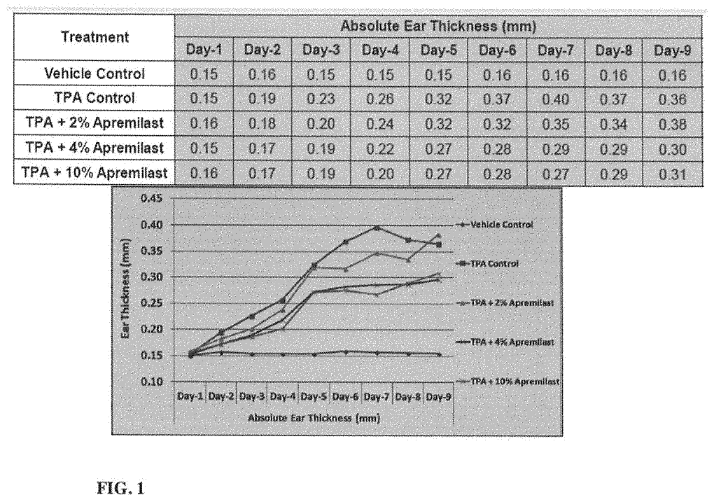

FIG. 2: Inflammatory Ear Edema

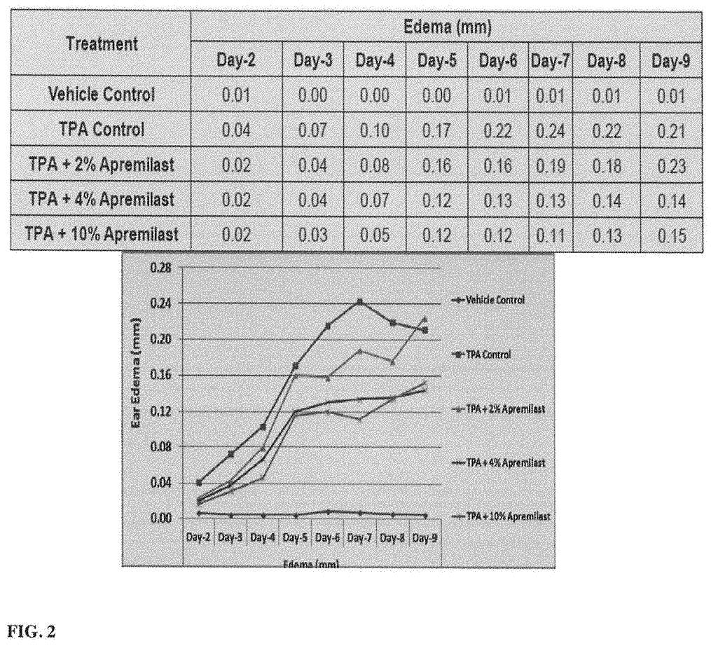

FIG. 3 illustrates % Inhibition of Ear Edema on topical treatment

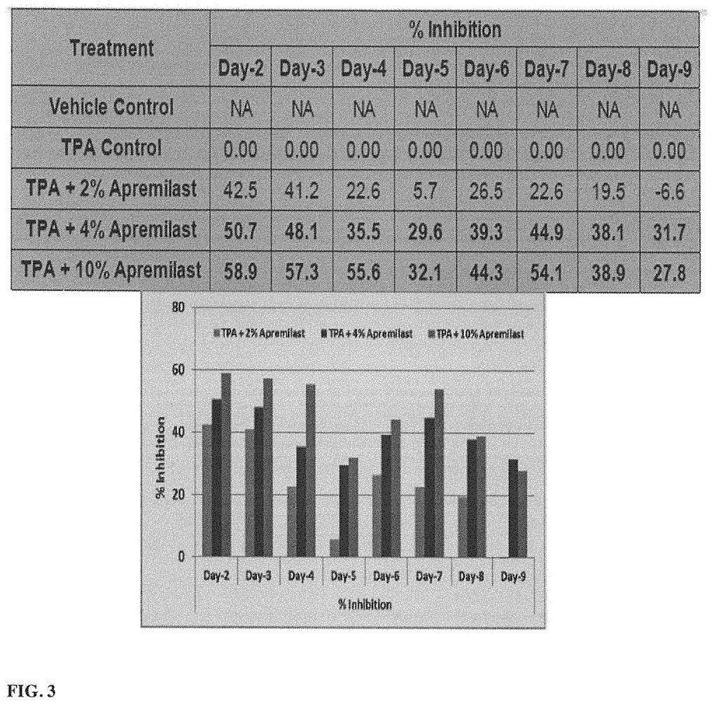

FIG. 4: 4 mm Ear Punch Biopsy Weight

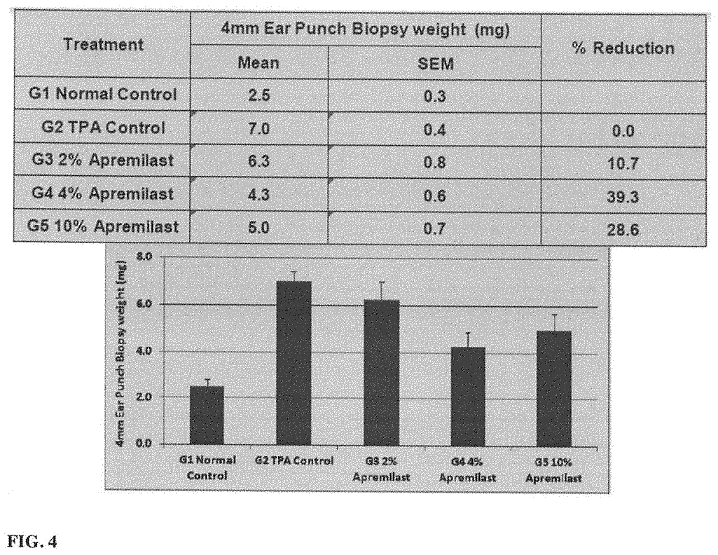

### DETAILED DESCRIPTION

The present invention relates to safe, effective and stable topical compositions of apremilast.

Accordingly, the present invention is directed to stable topical compositions comprising a therapeutically effective amount of apremilast, a pharmaceutically acceptable carrier and one or more pharmaceutically acceptable excipients.

The term “stable” as used herein refers to physical stability and/or chemical stability of the active agent in a topical composition, wherein changes in the drug assay values and/or impurities content are less than about 10%, during stability study storage of the composition at 25° C. and 60% relative humidity (RH), or 30° C. and 65% RH, or 40° C. and 75% RH, for durations such as 3, 6, 12, 18, or 24 months.

The term “topical”, as used herein, refers to a composition meant for application to the skin, nail, or mucosal tissue.

The term “apremilast”, as used herein, includes apremilast and its salts, polymorphs, hydrates, solvates, prodrugs, chelates, and complexes.

In one embodiment, the invention is directed to topical compositions comprising a therapeutically effective amount of apremilast, a pharmaceutically acceptable carrier and one or more pharmaceutically acceptable excipients.

A “therapeutically effective amount” is an amount necessary to palliate at least one symptom of psoriasis, dermatosis, eczema, rosacea, acne vulgaris, allergies, contact and atopic dermatitis, pruritus, seborrhea, skin cancers, inflammation or other associated skin conditions. For example, a therapeutically effective amount is sufficient to treat (i.e. alleviate or reduce) at least one of: itching/scratching, redness, inflammation, cracking, scaling, bleeding, etc. The topical composition of the present invention comprises apremilast in an amount from about 0.01% w/w to about 10% w/w of the total composition.

The term “pharmaceutically acceptable” means approved by a regulatory agency of the Federal or state government or listed in the U.S. Pharmacopeia or other generally recognized pharmacopeia for use in animals, and more particularly in humans.

The term “carrier” denotes organic or inorganic ingredients, natural or synthetic, with which an active ingredient is combined to facilitate application of a composition. Suitable carrier materials include any carrier or vehicle commonly used as a base for solutions, dispersions, emulsions, gels, creams, ointment, lotions, pastes, spray or foams, for topical administration. Examples include emulsifying agents, inert carriers including hydrocarbon bases, emulsifying bases, non-toxic solvents or water-soluble bases. any suitable liquid or gel-based carriers are well-known in the art. The carrier should be able to dissolve or disperse an active at an effective level, optionally with the aid of non-toxic surfactants. Examples include water, physiological salt solutions, alcohols (e.g., methanol, ethanol, propanol, or butanol), glycerol, glycols (e.g., ethylene glycol, propylene glycol, or ethoxydiglycol), polyethylene glycol (e.g., MW 400 to 20,000), water-alcohol/glycol blends, and the like. Suitable carriers and diluents for certain embodiments include, for example, water, saline, isotonic saline solutions, for example, phosphate-buffered saline, aqueous dextrose, glycerol, ethoxydiglycol, dimethyl sulfoxide (DMSO), and the like, or combinations thereof.

Suitable carriers further include aqueous and oleaginous carriers such as, for example, white petrolatum, isopropyl myristate, lanolin or lanolin alcohols, mineral oil, fragrant or essential oil, nasturtium extract oil, sorbitan mono-oleate, cetostearyl alcohol (together or in various combinations), and detergents (e.g., polysorbates (Tweens) such as polysorbate 20, 40, 60, or 80; polyoxyl stearate; or sodium lauryl sulfate). One or more carrier materials can be mixed with water to form a lotion, gel, cream, semi-solid composition, or the like. Other suitable carriers include water-in-oil or oil-in-water emulsions and mixtures of emulsifiers and emollients with solvents such as sucrose stearate, sucrose cocoate, sucrose distearate, mineral oil, propylene glycol, 2-ethyl-1,3-hexanediol, polyoxypropylene-15-stearyl ether, water, or combinations thereof. For example, emulsions containing water, glycerol stearate, glycerin, mineral oil, synthetic spermaceti, cetyl alcohol, or combinations thereof, may be used.

The amounts of carrier may be about 5% to about 99% of the total weight of the composition.

As a non-limiting example, a pharmaceutically acceptable carrier may comprise water, glycerin, petrolatum, stearic acid, glycol stearate, dimethicone, isopropyl isostearate, tapioca starch, cetyl alcohol, glyceryl stearate, magnesium aluminum silicate, carbomer, ethylene brassylate, triethanolamine, disodium EDTA, phenoxyethanol, methyl paraben, propyl paraben, ethanol, bio-polymers (e.g., sodium hyaloronate), liposomes, nano- and micro-particulate carriers, and/or titanium dioxide.

In embodiments, compositions of the present application include excipients including, but not limited to, emulsifiers, coemulsifiers, permeation or penetration enhancers, solvents, co-solvents, emollients, propellants, antioxidants, preservatives, buffering agents, gelling or thickening agents, polymers, plasticizers, film-formers, surfactants, soothing agents, pH modifiers, solubilizers, stabilizers, humectants, moisturizers, oily bases, and the like.

The term “emollient”, as used herein, refers to a substance that helps retain skin moisture and also helps control the rate of evaporation and the tackiness of the composition. Additionally, emollients provide a softening or soothing effect on the skin surface. Suitable examples of emollients are selected from the group consisting of fatty acid triglycerides such as mixtures of caprylic and capric triglycerides (e.g., Crodamol™), palmitic triglyceride, oleic triglyceride, caprylic triglyceride, capric triglyceride, and linoleic triglyceride; fatty acid esters such as isopropyl myristate, isopropyl palmitate, dibutyladipate, and dibutyl phthalate; polyhydric alcohols such as propylene glycol, butylene glycol, polyethylene glycol, glycerol, and sorbitol; fatty acids such as oleic acid and stearic acid; oils such as mineral oil, lanolin oil, coconut oil, cocoa butter, olive oil, jojoba oil, and castor oil; cyclomethicone; hydrogenated lanolin; waxes; lecithin; or mixtures thereof. Preferably, the emollient is selected from the group consisting of fatty acid triglycerides, fatty acid esters, and polyhydric alcohols.

The term “propellant”, as used herein, refers to the substance that helps in propelling the composition out of the container. Suitable examples of propellants are selected from the group consisting of conventional, non-ozone depleting hydrocarbon propellants, including propane, butane, isobutane, cyclopropane, 1,1,1,2-tetrafluorethane, 1,1,1,2,3,3,3-heptafluoropropane, 1,1-difluoroethane, 1,1,1,3,3,3-hexafluoropropane, and mixtures thereof; fluorocarbon gas; and liquefied petroleum gas.

The term “emulsifying agents” as used herein includes any a wide variety of cationic, anionic, zwitterionic, and amphoteric surfactants that are known in the art. Non-limiting examples of anionic emulsifying agents include sodium lauryl sulfate, alkyl isethionates, alkyl and alkyl ether sulfates and salts thereof, alkyl and alkyl ether phosphates and salts thereof, alkyl methyl taurates, and soaps (e.g., alkali metal salts and sodium or potassium salts) of fatty acids.

Examples of amphoteric and zwitterionic emulsifying agents include those which are broadly described as derivatives of aliphatic secondary and tertiary amines in which the aliphatic radical can be straight or branched chain, wherein one of the aliphatic substituents contains from about 8 to about 22 carbon atoms and one contains an anionic water solubilizing group, e.g., carboxy, sulfonate, sulfate, phosphate, or phosphonate. Specific examples include alkylimino acetates, iminodialkanoates and aminoalkanoates, imidazolinium and ammonium derivatives. Other suitable amphoteric and zwitterionic emulsifying agents include betaines, sultaines, hydroxysultaines, alkyl sarcosinates, and alkanoylsarcosinates.

Nonionic emulsifying agents include those that can be broadly defined as condensation products of long chain alcohols, e.g., C8-30 alcohols, with sugar or to starch polymers, i.e., glycosides. Various sugars include, but are not limited to, glucose, fructose, mannose, and galactose, and various long chain alcohols include, but are not limited to, decyl alcohol, cetyl alcohol, stearyl alcohol, lauryl alcohol, myristyl alcohol, oleyl alcohol, and the like.

Other useful nonionic emulsifying agents include condensation products of alkylene oxides with fatty acids such as alkylene oxide esters of fatty acids. Other nonionic surfactants are the condensation products of alkylene oxides with 2 moles of fatty acids such as alkylene oxide diesters of fatty acids.

Silicone emulsifying agents are typically organically modified organopolysiloxanes, sometimes called silicone surfactants. Useful silicone emulsifying agents include dimethicone copolyols. These materials are polydimethylsiloxanes, which have been modified to include polyether side chains such as polyethylene oxide chains, polypropylene oxide chains, mixtures of these chains, and polyether chains containing moieties derived from both ethylene oxide and propylene oxide.

The amounts of emulsifier may be about 0.25% to about 45% of the total weight of the composition.

The term “co-emulsifiers” or secondary emulsifying agents include polyoxylglycerides such as oleoylmacrogolglycerides (Labrafil® M 1944CS), linoleoylmacrogolglycerides (Labrafil® M 2125CS), caprylocaproylmacrogolglycerides (Labrasol®), cetyl alcohol (and) ceteth-20 (and) steareth-20 (Emulcire™ 61 WL 2659), glyceryl stearate (and) PEG-75 stearate (Gelot® 64), and any mixtures thereof.

A “permeation enhancer” or “penetration enhancer” is a component used to enhance the penetration rate of drugs through the skin or mucous membrane, such as by temporarily diminishing the impermeability of the skin or membrane. Permeation enhancers have also been called “accelerants” and “absorption promoters.” There are numerous penetration enhancers that can be used. Various useful permeation enhancers include, for example: polyols and esters, including polyethylene glycol, polyethylene glycol monolaurate, and butanediol; sulfoxides, including dimethylsulfoxide and decylmethylsulfoxide; ethers, including diethylene glycol monoethyl ether (e.g., Transcutol® P) and diethylene glycol monomethyl ether; fatty acids, including lauric acid, oleic acid, and valeric acid; fatty acid esters, including isopropyl myristate, isopropyl palmitate, methyl propionate, and ethyl oleate; nitrogenous compounds including urea, dimethyl acetamide, dimethylformamide 2-pyrrolidone, ethanolamine, methyl-2-pyrrolidone, diethanolamine, and triethanolamine; terpenes; alkanones; organic acids, including salicylic acid, citric acid, and succinic acid; and any mixtures thereof. Further, one or more surfactants can also be used as a permeation or penetration enhancer.

A permeation enhancer can be used in concentrations ranging from about 0.001-15%, preferably, about 0.05-12%, more preferably, about 3-10%, of the total weight of the composition.

The term “preservative” refers to a natural or synthetic chemical that is added to products to prevent decomposition by microbial growth or by undesirable chemical changes. Preservatives can desirably be incorporated into a composition for protecting against the growth of potentially harmful microorganisms. While microorganisms tend to grow in an aqueous phase, microorganisms can also reside in a hydrophobic or oil phase. Suitable preservatives for compositions of the present invention include, but are not limited to, methylparaben, propylparaben, benzyl alcohol, chlorocresol, benzalkonium chloride, cetrimonium chloride, sodium edetate, boric acid, and any mixtures thereof.

The amount of the preservative may be about 0.25% to about 25% of the total weight of the composition.

“Antioxidants” refers to substances which inhibit oxidation or suppress reactions promoted by oxygen or peroxides. Antioxidants, especially lipid-soluble antioxidants, can be absorbed into the cellular membrane to neutralize oxygen radicals and thereby protect the membrane. Suitable antioxidants for compositions of the present invention include, but are not limited to, ascorbic acid (vitamin C), glutathione, lipoic acid, uric acid, carotenes, α-tocopherol (vitamin E), ubiquinol, butylated hydroxyanisole, butylated hydroxytoluene, sodium benzoate, sodium thiosulphate, propyl gallate (PG, E310), and tertiary-butylhydroquinone.

The amounts of antioxidant may be about 0.01% to about 20%, of the total weight of the composition.

“Solvent” refers to components that aid in the dissolution of the drug in the formulation. Solvents serve to maintain a solution of the drug in the composition. Some solvents can also enhance percutaneous penetration of drug and/or act as humectants. For steroid drugs, solvents can include water-immiscible substances such as fatty esters of natural fatty acids, triglycerides of animal or vegetable, medium chain triglycerides, mixtures of mono-, di- and/or triglycerides, waxes, hydrogenated vegetable oils, and mixtures thereof. Some specific examples include castor oil, lanolin oil, citrate triisocetyl triglycerides having 10-18 carbon atoms, caprylic/capric triglycerides, coconut oil, corn oil, cottonseed oil, linseed oil, oil of mink, olive oil, palm oil, sunflower oil, nut oil, diethylene glycol monoethyl ether, diethylene glycol monomethyl ether, saturated paraffin oils, light or heavy mineral oils, vegetable oils or glycerides, and the like.

The term “plasticizer” as used herein is a substance that aids the composition in forming a flexible, adherent film on the skin. Suitable plasticizers are selected from the group consisting of citric acid esters, dimethyl isosorbide, castor oil, propylene glycol, polyethylene glycol, glycerol, oleic acid, citric acid, adipic acid, phosphate esters, fatty acid esters, glycol derivatives, hydrocarbons and their derivatives, butanediol polyesters, diethyl phthalate, dibutyl phthalate, chlorinated paraffins, and mixtures thereof.

The term “film-former” as used herein is a substance that forms a stable film on a topical surface when applied. Suitable film-formers are selected from the group consisting of acrylic polymers or copolymers such as methacrylic acid copolymers; cellulose derivatives such as cellulose acetate, hydroxypropyl methyl cellulose, hydroxy ethyl cellulose, methyl cellulose, and ethyl cellulose; polyvinyl acetate; polyvinyl alcohol; povidone; povidone vinyl acetate; and mixtures thereof. These film-formers can partially dissolve on exposure to moisture from the skin or air, the dissolution resulting in the formation of a porous film. This porosity can be enhanced by including additional water-soluble additives. The water-soluble additive is preferably propylene glycol, sodium lauryl sulphate, poloxamers, polyoxyl 35 castor oil, polyoxyl 40 hydrogenated castor oil, cetomacrogol, polyethylene glycol, transcutol, or mixtures thereof.

Suitable pH-adjusting agents are selected from the group consisting of pharmaceutically acceptable organic or inorganic acids or bases such as sodium hydroxide, tromethamine, hydrochloric acid, inorganic oxides, inorganic salts of weak acids, and mixtures thereof.

Optionally, the compositions of the present invention may further contain one or more additional active ingredients, such as an antibacterial, disinfectant, antifungal, analgesic, anti-inflammatory, emollients, local anaesthetics and the like.

Additional active agents may include, but are not limited to methotrexate, 6-MP, azathioprine sulphasalazine, mesalazine, olsalazinechloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolatemofetil, leflunomide, teriflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNFa or IL-1 (e.g., IRAK, NIK, IKK, p38 or MAP kinase inhibitors), IL-Iβ converting enzyme inhibitors, TNFα converting enzyme (TACE) inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors and the derivatives p75 TNFRIgG (ENBREL™ and p55 TNFRIgG (Lenercept)), sIL-1RI, sIL-1RII, sIL-6R), anti-inflammatory cytokines (e.g., IL-4, IL-10, IL-12, IL-13 and TGFP), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, human recombinant, tramadol HCl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline HCl, sulfadiazine, oxycodone HCl/acetaminophen, olopatadine HCl, misoprostol, naproxen sodium, omeprazole, cyclophosphamide, rituximab, IL-1 TRAP, MRA, CTLA4-IG, IL-18 BP, anti-IL-18, Anti-IL15, BIRB-796, SC10-469, VX-702, AMG-548, VX-740, Roflumilast, IC-485, PSORIASIS C-801, and Mesopram. Suitable antimicrobials include, but are not limited to, quaternary ammonium salts such as benzalkonium chloride.

Some of the excipient substances described above can have more than one function in a formulation. For example, a substance can be both a solvent and a penetration enhancer, or both a solvent and a carrier. The categorizations of materials described above are not to be construed as limiting or restricting in any manner.

In another embodiment, the present application provides pharmaceutical compositions comprising apremilast as the active agent, in any dosage form suitable for topical administration. Preferably, the compositions are in a form such as a solution, suspension, dispersion, emulsion, cream, ointment, gel, lotion, foam, paste or spray.

Various topical delivery systems are known and can be used to administer a composition of the present invention, e.g., encapsulation in liposomes, microparticles, microcapsules, etc.

For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure.

In embodiments, compositions may be in the form of an emulsion. The emulsion can be in the form of an oil-in-water type of emulsion or a water-in-oil type of emulsion. An aqueous-based emulsion, such as an oil-in-water emulsion, frequently has lower viscosity than other emulsion types and exhibits appreciable storage stability. Generally, oil-in-water emulsions have better skin feel properties, when applied to the skin, as these give sensations similar to an aqueous material.

Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle.

“Creams” herein refers to viscous liquids or semisolid emulsions, either oil-in-water or water-in-oil. Cream bases are water-washable, and comprise an oil phase, an emulsifier, and an aqueous-phase. Water-in-oil creams may be formulated by using a suitable emulsifying agent with properties similar, but not limited, to those of the fatty alcohols such as cetyl alcohol or cetostearyl alcohol and to emulsifying wax. Oil-in-water creams may be formulated using an emulsifying agent such as cetomacrogol emulsifying wax. Suitable properties include the ability to modify the viscosity of the emulsion and both physical and chemical stability over a wide range of pH. The water soluble or miscible cream base may contain a preservative system and may also be buffered to maintain an acceptable physiological pH.

“Ointments” herein refer to semisolid preparations that include the active incorporated into a fatty, waxy, or synthetic base. Ointments are typically based on petrolatum or other petroleum derivatives. The specific ointment base to be used, as will be appreciated by those skilled in the art, is one that will provide for suitable drug delivery and other desired characteristics such as emolliency or the like.

“Gels” herein refer to clear, sticky, jelly-like semisolids or solids prepared from high molecular weight polymers in an aqueous or alcoholic base. Alcoholic gels are often drying and cooling. Non-alcoholic gels are more lubricating. In some embodiments, gel formulations will include the same or similar ingredients as a solution or dispersion, with the addition of a gelling agent.

“Lotions” herein refer to liquid or semi liquid preparations in which solid particles, including the active agent(s), are present in a water or alcohol base. Lotions are usually suspensions of solids, and can include a liquid oily emulsion of the oil-in-water type. Lotions are often desirable formulations because of the ease of applying a more fluid composition. Lotions will typically contain suspending agents to produce better dispersions as well as compounds useful for localizing and holding the active agent in contact with the skin, e.g., methylcellulose, sodium carboxymethylcellulose, or the like.

“Foam” herein refer to preparations formulated to be delivered from a pressurized aerosol canister, via a suitable applicator, using inert propellants. Suitable excipients for the formulation of the foam base include, but are not limited to, propylene glycol, emulsifying wax, cetyl alcohol, and glyceryl stearate.

“Pastes” are semisolid dosage forms in which the active agent is suspended in a suitable base. Depending on the nature of the base, pastes are divided between fatty pastes or those made from a single-phase aqueous gel. The base in a fatty paste is generally petrolatum, hydrophilic petrolatum, or the like. The pastes made from single-phase aqueous gels generally incorporate carboxymethylcellulose or the like as a base.

“Sprays”, as used herein, means to dispense the composition as a mass or jet of droplets from a dispensing system.

In an aspect, topical application of compositions of the present application forms a depot on the skin without forming an occlusive film, thereby extending the duration of active agent action while allowing ‘breathing’ of the skin.

In yet another embodiment, the present application provides uses of topical compositions comprising apremilast as the active agent, for prophylaxis, amelioration, or treatment of skin diseases or disorders such as psoriasis, dermatosis, eczema, rosacea, acne vulgaris, allergies, contact and atopic dermatitis, pruritus, seborrhea, skin cancers, inflammationand other associated skin conditions.

In an embodiment, topical compositions of the present application are useful in the management of psoriasis, and further can provide a moisturizing and/or soothing effect at the site of application to the skin. In an embodiment, the compositions reduce the dryness that accompanies the build-up of skin in psoriatic plaques. In yet another embodiment, the compositions can be applied directly to the psoriatic lesions or dermatosis and can help reduce inflammation, remove built-up scale, reduce skin turnover, and/or clear affected skin of plaques.

In an embodiment, the invention provides relief of skin irritation, discomfort, itch and other symptoms caused by a variety of conditions, including but not limited to psoriasis, dermatosis, eczema, rosacea, acne vulgaris, allergies, contact and atopic dermatitis, pruritus, seborrhea, skin cancers, inflammation and other associated skin conditions.

In another aspect, the present invention sets forth a safe and commercially viable process for preparation of therapeutically effective topicalcompositions of apremilast that are sufficiently stable to provide an acceptable shelf life.

Thus, in another embodiment, the present invention is directed to methods of preparation of topical compositions comprising apremilast, and effective in the treatment of psoriasis, dermatosis, eczema, rosacea, acne vulgaris, allergies, contact and atopic dermatitis, pruritus, seborrhea, skin cancers, inflammation and other associated skin conditions. The compositions of the present invention may be prepared through any of the processes and techniques known in the art. The inventor have designed different formulation procedures, and varieties of excipients of oil and aqueous phases, surfactants and solubility enhancers, and emulsifiers in order to develop stable, uniform, and cosmetically acceptable compositions.

The principles, preferred embodiments, and modes of operation of the present invention have been described in the foregoing specification. The invention which is intended to be protected herein, however, is not to be construed limited to the particular forms disclosed, since these are to be regarded as illustrative rather than restrictive. Variations and changes may be made by those skilled in the art, without departing from the spirit of the invention.

The following examples further illustrate the invention but should not be construed as in any way limiting its scope. In particular, the processing conditions are merely exemplary and can be readily varied by one of ordinary skill in the art.

### EXAMPLES

### Example 1: Topical Composition of Apremilast

| Component | % w/w |
|---|---|
| Apremilast | 2.0 |
| Ethyl alcohol | 90.3 |
| Isopropyl myristate | 7.30 |
| Sodium lauryl sulfate | 0.1 |
| Undecylenic acid | 0.3 |

### Procedure:

a) Apremilast is dissolved into a portion of ethyl alcohol while stirring.
b) Isopropyl myristate and undecylenic acid is added while stirring into the solution of step (a).
c) The remaining quantity of ethyl alcohol is added into the solution of step (b) and mixed.

### Example 2: Topical Composition of Apremilast

| Component | % w/w |
|---|---|
| Apremilast | 4.0 |
| Ethyl alcohol | 70.0 |
| Water | 20.5 |
| Sodium iodide | 3.7 |
| Potassium iodide | 1.4 |
| Sodium thiosulphate | 0.4 |

### Procedure:

a) Apremilast is dissolved into a portion of ethyl alcohol while stirring.
b) Iodides and sodium thiosulphate are solubilized in water
c) The solution obtained in step (b) is added while stirring into the solution of step (a).
d) The volume is made up to the required quantity by adding ethyl alcohol and water to the solution of step (c) and mixed.

### Example 3: Topical Composition of Apremilast

| Component | % w/w | % w/w |
|---|---|---|
| Apremilast | 2.0 | 4.0 |
| Ethyl alcohol | 74.74 | 6.15 |
| Isopropyl myristate | 12.0 | 10.0 |
| Transcutol | 3.0 | 9.0 |
| Mineral oil | 7.26 | 69.85 |
| Acrylic polymer | 1.0 | 1.0 |

### Procedure:

a) Apremilast is dissolved in a mixture isopropyl myristate, mineral oil and transcutol with stirring.
b) Acrylic polymer is dissolved in ethanol.
c) The solution obtained in step (b) is added while stirring into the solution of step (a).
d) The volume is made up to the required quantity by adding ethyl alcohol and mineral oil to the solution of step (c) and mixed.

### Example 4: Topical Composition of Apremilast

| Component | % w/w | % w/w |
|---|---|---|
| Apremilast | 4.0 | 2.0 |
| Polyethylene glycol/Ethylene | 7.5 | 7.5 |
| glycol palmitostearate | | |
| Mineral oil | 7.06 | 7.06 |
| Oleyl polyoxyglycerides | 2.94 | 2.94 |
| Diethylene glycol monoethyl ether | 5.0 | 5.0 |
| Propyl paraben | 0.8 | 0.8 |
| Methyl paraben | 0.2 | 0.2 |
| Butylated hydroxyl toulene | 0.05 | 0.05 |
| Hydroxy ethyl cellulose | 0.1 | 0.00 |
| Xanthum gum | 0.00 | 0.01 |
| Water | 72.35 | 74.35 |

### Procedure:

a) Polyethylene glycol and ethylene glycol palmitostearate, oleoyl polyoxyglycerides, and mineral oil are mixed and heated to about 50-70° C.
b) Propylparaben, methylparaben, and butylated hydroxytoluene are mixed with the liquid of (a) with continuous stirring at about 50-70° C.
c) Apremilast is mixed with diethylene glycol monoethyl ether.
d) The material of (c) is mixed with the material of (b).
e) Hydroxyethyl cellulose or xanthan gum is dissolved in water.
f) The oily phase of (d) is added slowly to the aqueous phase of (e), or vice versa, at about 50-70° C. with continuous stirring.
g) The mixture of (f) is homogenized and allowed to cool to ambient temperature.

### Example 5: Topical Composition of Apremilast

| Component | % w/w | % w/w |
|---|---|---|
| Apremilast | 2.0 | 4.0 |
| Ethyl alcohol | 49.0 | 47.25 |
| Polyethylene glycol 400 | 48.85 | 48.75 |
| Sodium Lauryl Sulphate | 0.1 | 0.1 |

### Procedure:

a) Apremilast was dissolved in a portion of ethyl alcohol while stirring.
b) Polyethylene glycol 400 was added while stirring into the solution of step (a).
c) Sodium lauryl sulfate was added while stirring into the solution of step (b).
d) The remaining quantity of ethyl alcohol was added into the solution of step (c) and mixed.

### Example 6: Topical Composition of Apremilast

| Component | % w/w | % w/w |
|---|---|---|
| Apremilast | 2.0 | 4.0 |
| Ethyl alcohol | 47.0 | 45.0 |
| Caprylic and capric triglycerides | 50.0 | 50.0 |
| (Crodamol ™) | | |
| Oleic acid | 0.85 | 0.85 |
| Sodium lauryl sulfate | 0.1 | 0.1 |

### Procedure:

a) Apremilast was dissolved in a portion of ethyl alcohol while stirring.
b) Crodamol™ was added while stirring into the solution of step (a).
c) Oleic acid was added while stirring into the solution of step (b)
d) Sodium lauryl sulfate was added while stirring into the solution of step (c).
e) The remaining quantity of ethyl alcohol was added into the solution of step (d) and mixed.

### Example 7: Topical Composition of Apremilast

| Component | % w/w | % w/w |
|---|---|---|
| Apremilast | 2.0 | 4.0 |
| Propylene Glycol | 50.95 | 50.95 |
| Ethyl alcohol | 47.0 | 45.0 |

### Manufacturing Process:

a) Apremilast was dissolved in a portion of ethyl alcohol while stirring.
b) Polyethylene glycol 400 was added while stirring into the solution of step (a).
c) The remaining quantity of ethyl alcohol was added into the solution of step (b) and mixed.

### Example 8: Pre-Clinical Studies Using Test Formulations

Efficacy of the test formulations was determined in studies using 12-O-Tetradecanoylphorbol-13-Acetate (TPA) induced ear inflammation in mice.

Principle:

Excessive proliferation of keratinocytes is a characteristic of psoriasis, and this cell type is a well reported target of therapy for this disease. In the present study, chronic skin inflammation was induced by repeated topical application of TPA, which is recognized by prolonged skin reaction & epidermal hyperplasia.

Animal:

The animal model used was Male C57BL/6 mice 6 to 8 weeks of age.

Protocol:
a) Animals were randomized based on body weight and divided into six different groups consisting of 8 animals.
b) 20 μL of TPA solution containing 2 μg of TPA in vehicle (2% DMSO and 98% methanol) was applied topically on both ventral and dorsal side (10 μL each side) of the right ear of all the animals except group Group G1 on day 0, 2, 4, 7 and 9.
c) Group G1 and Group G2 were topically treated with placebo gel formulation on right ear.
d) Groups G3, G4 and G5 were topically treated with test compositions comprising apremilast (2% w/w, 4% w/w and 10% w/w concentration).
e) Group G6 was topically treated with 0.1% Betamethasone valerate cream.
f) All the formulations were uniformly applied, 25 mg each (50 mg per animal), in anterior and posterior region of the right ear daily from day 0 to day 10.

### Observations:

a) Alternate day topical TPA application on ear lead to ear inflammation.
b) Maximum ear thickness and ear edema was observed on day-7.
c) Psoriatic skin was observed from day-4 onwards.
d) Daily topical application of apremilast (in test formulation) at the tested concentration (2%, 4% and 10%) was found to be effective against ear inflammation.
e) Dose-dependent increase in efficacy was observed between 2% and 4%; however, saturation of efficacy was observed at 10% concentration of apremilast.
f) Application of test formulation containing 2% apremilast showed poor reduction on ear edema which tapered off by day-9.
g) Test formulation containing 4% and 10% apremilast showed similar efficacy in reducing ear inflammation.
h) The punch biopsy weight measurements also showed similar findings.

While the present disclosure has been illustrated and described with respect to a particular embodiment thereof, it should be appreciated by those of ordinary skill in the art that various modifications to this disclosure may be made without departing from the spirit and scope of the present disclosure.

What is claimed is:

1. A pharmaceutical composition suitable for topical administration, comprising apremilast, or a pharmaceutically acceptable salt thereof, wherein said apremilast is present in the range of 0.01% to 20% by weight.

2. (canceled)

3. The pharmaceutical composition of claim 1, wherein the composition is in the form of a solution, suspension, dispersion, emulsion, cream, ointment, gel, lotion, foam, paste orspray.

4. A pharmaceutical composition suitable for topical administration, comprising:
i) Apremilast, in the range of 0.01% to 20% by weight;
ii) at least one pharmaceutically acceptable carrier; and
iii) optionally, one or more pharmaceutically acceptable excipients.

5. (canceled)

6. The pharmaceutical composition of claim 4, wherein the composition is in the form of a solution, suspension, dispersion, emulsion, cream, ointment, gel, lotion, foam, paste or spray.

7. The pharmaceutical composition of claim 4, wherein the composition further comprises emulsifiers, coemulsifiers, permeation or penetration enhancers, solvents, co-solvents, emollients, antioxidants, propellants, preservatives, buffering agents, gelling or thickening agents, polymers, plasticizers, film-formers, surfactants, soothing agents, pH modifiers, solubilizers, stabilizers, humectants, moisturizers, oily bases, or mixtures thereof.

8. The pharmaceutical composition of claim 4, wherein the composition further comprises one or more additional active ingredients selected from the group consisting of antibacterial agents, disinfectant agents, antifungal agents, analgesics, anti-inflammatory agents, emollients, or a local anesthetics.

9. A method treating or preventing a dermatological diseases or disorders comprising topically administering to the site of the dermatological disease or disorder a composition according to claim 1, in an amount effective to treat or prevent such diseases or disorders.

10. The method of claim 9, wherein the dermatological disease or disorder is selected from the group consisting of psoriasis, dermatosis, eczema, rosacea, acne vulgaris, allergies, contact and atopic dermatitis, pruritus, seborrhea, skin cancers, inflammations and combinations thereof.

11. A method treating or preventing a dermatological diseases or disorders comprising topically administering to the site of the dermatological disease or disorder a composition according to claim 4, in an amount effective to treat or prevent such diseases or disorders.

12. The method of claim 11, wherein the dermatological disease or disorder is selected from the group consisting of psoriasis, dermatosis, eczema, rosacea, acne vulgaris, allergies, contact and atopic dermatitis, pruritus, seborrhea, skin cancers, inflammations and combinations thereof.
